(19) United States
(12) Patent Application Publication
(10) Pub. No.: US 2013/0059842 A1
(43) Pub. Date: Mar. 7, 2013

(54) MANUFACTURING OF QUICK RELEASE PHARMACEUTICAL COMPOSITONS OF WATER INSOLUBLE DRUGS AND PHARMACEUTICAL COMPOSITIONS OBTAINED BY THE PROCESS OF THE INVENTION

(71) Applicant: Nycomed Danmark ApS, Roskilde (DK)

(72) Inventor: Poul Egon Bertelsen, Vanlose (DK)

(73) Assignee: NYCOMED DANMARK APS, Roskilde (DK)

(21) Appl. No.: 13/666,278

(22) Filed: Nov. 1, 2012

Related U.S. Application Data
(62) Division of application No. 11/631,091, filed on Dec. 28, 2006.

(30) Foreign Application Priority Data
Jun. 29, 2004 (DK) PA 2004 01021

(51) Int. Cl. A61K 31/542; A61P 29/00

(52) U.S. Cl. 514/226.5

(57) ABSTRACT

It has been found that pharmaceutical compositions comprising water insoluble drugs can be manufactured and formulated in a manner ensuring fast dissolution in gastric fluid. Advantageously, the manufacturing process provides a significantly improved stability, thus resulting in compositions that may have a longer shelf life than conventionally formulated and processed drugs.

### FIELD OF INVENTION

The present invention relates to the field of pharmaceutical formulation science, in particular with respect to methods of improving the solubility and dissolution of water-insoluble drugs. The present invention is especially focused on compositions comprising a drug substance belonging to the class of drug substances normally denoted NSAID's (non-steroidal anti-inflammatory drug). In particular NSAID's with low solubility in water and gastric fluid, and with stability problems are of interest. An example of such an NSAID is lornoxicam. However, other drug substances having a low solubility in acidic medium and/or a pKₐ below about 5.5, may as well be suitable for being formulated in a composition according to the invention. The present invention provides oral dosage forms with a significantly improved stability.

### BACKGROUND

Fast absorption of drugs into the circulating blood is generally required in managing pain relieves. Therefore, for oral dosage forms it is of utmost importance to have the drug dissolved, completely or partly, already when present in the gastric fluid. Thus, in the event where the drug is not absorbed from the gastric mucosa, it may be ready for being absorbed already when entering the upper intestinal tract, such as duodenum. Duodenum itself has a limited amount of liquid, thus resulting in slow dissolution of the drug in duodenum, although the weak acid may be more soluble in the intestinal fluid.

Several approaches have been reported for the manufacturing and the formulation of oral dosage forms of drugs that are substantially water-insoluble and are weak acids so as to achieve quick in vitro dissolution in gastric fluid.

Some references have taken the approach of improving the solubility of an NSAID by forming an inclusion complex with cyclodextrin. WO 9641646 relates to a parenteral formulation of lornoxicam formulated as an inclusion complex with cyclodextrin. The medicament is either present as a powder for reconstitution or as a solution. All examples imply a solvating step and the reference is silent of preparing the formulation without the use of water.

Likewise, WO 9532737 relates to an inclusion complex of an NSAID, such as lornoxicam and a cyclodextrin. The manufacturing process includes a wetting step so the reference does necessarily imply the use of water in the formulation process.

Other references have taken other approaches for improving the solubility of an NSAID. For example, it is steadily reported that such water insoluble drugs need to be formulated together with an alkaline substance. It is further reported that the manufacturing process should include contacting the drug-containing and alkaline containing powder with an aqueous medium to form a particulate composition.

EP 1109534 B1 discloses a formulation comprising a therapeutically and/or prophylactically active substance, which has a solubility of at the most about 0.1% w/v in 0.1 N hydrochloric acid at room temperature, the composition being based on a powder that comprises the active substance and an alkaline substance and which are being contacted with an aqueous medium to form a particulate composition. Such compositions have a dissolution rate in 0.1 N hydrochloric acid such that at least 50% of the drug is present on dissolved form within the first 20 minutes of dissolution testing. Importantly, it is stated that the fast dissolution rate disclosed in this patent is not achieved if the active substance and the alkaline substance are processed under conditions where an aqueous contact between the two components does not take place (i.e. under anhydrous conditions).

WO 9912524 relates to a modified release multiple-unit formulation where the active substance is an NSAID. The formulation is characterised in having two fractions of multiple-units where one fraction is fast-releasing and the other is slow releasing. The fast releasing fraction corresponds to the fast release formulation of reference EP 1109534 (WO 15195) cited above.

JP 3240729 and EP 792147 also relate to formulations wherein the active compound is granulated together with an alkaline substance using an aqueous solution.

However, until now it is the understanding that the use of aqueous solutions during the manufacturing process impose separate problems for drugs that are unstable in the presence of water and an alkaline substance. On the other hand, the prior art clearly teaches that wet-granulation is needed to provide a fast in vitro dissolution. Therefore, there is a need for providing formulations, still possessing fast dissolution in hydrochloric acid, while at the same time exhibiting a good stability.

### SUMMARY OF INVENTION

Now provided is a pharmaceutical composition, intended for oral administration, and manufactured by a process utilising a minimum of liquid, preferably without utilising liquid at all and utilising intensive mixing in the form of co-milling or the equivalent so as to provide close physical contact between the active drug substance and a dissolution helper (alkaline substance).

Though the oral dosage form was manufactured without using any liquid, the resulting batches had a water content on the same level as batches produced by means of wet-granulation.

Surprisingly, the batches produced according to the invention had a significantly improved stability irrespective of the water content.

Remarkably, the batches resulting from the co-milling process still processed a fast in vitro dissolution: The active drug substance has a fast in vitro dissolution rate at conditions simulating the gastric fluid, so that that at least 50% of the active drug substance is dissolved within the first 20 minutes of in vitro dissolution testing.

Accordingly, in a first aspect the invention is directed to a process for manufacturing a pharmaceutical composition as well as pharmaceutical compositions obtainable therefrom:

The process comprises the steps of:
a) providing an active drug substance, which has a solubility at room temperature less than 0.1% w/v in 0.1 N hydrochloric acid or has a pKₐ value less than 5.5; and
b) providing one or more alkaline substance(s); and
c) mixing said active drug substance and said alkaline substance by co-milling without adding a liquid, and optionally
d) admixing one or more pharmaceutically acceptable excipients and optionally
e) compressing said mixture c) or d) into a tablet.

Thus, present invention is directed not only to methods for manufacturing of pharmaceutical compositions, but also to stable pharmaceutical compositions.

### DETAILED DESCRIPTION OF THE INVENTION

It has surprisingly been found that pharmaceutical compositions comprising water insoluble drugs can be manufactured and formulated in a manner ensuring fast dissolution of the active drug substance in the gastric fluid, while also providing conditions for improving the stability with respect to the active drug substance. In the present invention the drug substance is not exposed, or at least only to a minimum extent, to any liquid or to any aqueous solution during the manufacturing process. The composition resulting from a process excluding water was until now believed to result in compositions with a lower water content than seen after wet-granulation. A low water content was expected to provide compositions potentially having longer shelf life than conventionally formulated and processed drugs. The water content in compositions manufactured according to the invention proved, however, to have a water content on the same level as compositions produced by means of wet-granulation which granulate was subsequently dried. Very surprisingly the present co-milling process provides a significantly improved stability despite of the water content. At the same instance the provided compositions display a fast in vitro dissolution, though the prior teaching emphasize that the active substance and the alkaline excipient should be contacted with water for providing a fast in vitro dissolution. Thus, for active substances sensitive to water, the process is advantageous.

Advantageously, the described co-milling process is economically profitable as the process requires fewer production steps. Especially, is a laborious wetting step and the subsequent expensive drying step of the prior manufacturing method are avoided. Furthermore, special requirements regarded dehumidification of the air at the production facilities may be avoided.

In a first aspect, the invention provides a process for manufacturing an oral dosage form having a fast dissolution of the active drug substance in gastric fluid; the process comprises the steps of:
a) providing an active drug substance, which has a solubility at room temperature less than 0.1% w/v in 0.1 N hydrochloric acid or has a pKₐ value less than 5.5; and
b) providing one or more alkaline substance(s); and
c) mixing said active drug substance and said alkaline substance by co-milling without adding a liquid, and optionally
d) admixing one or more pharmaceutically acceptable excipients and optionally
e) compressing said mixture c) or d) into a tablet.

In a second aspect, the invention provides a process for manufacturing an oral dosage form having a fast dissolution of the active drug substance in gastric fluid; the process comprises the steps of:
a) providing an active drug substance, which has a solubility at room temperature less than 0.1% w/v in 0.1 N hydrochloric acid or has a pKₐ value less than 5.5; and
b) providing one or more alkaline substance(s); and
c) milling said alkaline substance without adding a liquid,
d) admixing said alkaline substance(s) obtained in c) with said active drug substance, and optionally
e) admixing one or more pharmaceutically acceptable excipients, and
f) compressing said mixture d) or e) into a tablet.

In a third aspect, the invention provides a process for manufacturing an oral dosage form having a fast dissolution of the active drug substance in gastric fluid; the process comprises the steps of:
a) providing an active drug substance, which has a solubility at room temperature less than 0.1% w/v in 0.1 N hydrochloric acid or has a pKₐ value less than 5.5; and
b) providing one or more alkaline substance(s); and
c) mixing said active drug substance and said alkaline substance and optionally one or more excipients by co-milling without adding a liquid, and optionally
d) admixing one or more pharmaceutically acceptable excipients, and optionally
e) compressing said mixture c) or d) into a tablet.

In a fourth aspect, the invention provides a process for manufacturing an oral dosage form having a fast dissolution of the active drug substance in gastric fluid; the process comprises the steps of:
a) providing an active drug substance, which has a solubility at room temperature less than 0.1% w/v in 0.1 N hydrochloric acid or has a pKₐ value less than 5.5; and b) providing one or more alkaline substance(s) having a solubility in water of at least 40 mg/ml and a mean particle size in the range of 1 to 400 μm, such as 1 to 300 μm, such as 5 to 200 μm; and
c) admixing said alkaline substance(s) obtained in b) with said active drug substance, and optionally
d) admixing one or more pharmaceutically acceptable excipients, and
e) compressing said mixture c) or d) into a tablet.

That is to say that the process is undertaken under dry conditions excluding the use of liquid. Such oral dosage forms are preferably in the form of a solid or a semi-solid.

In another aspect, the invention provides, in general terms, an oral dosage form with quick release of the active ingredient in that the oral dosage form comprises the active drug substance as defined herein in close physical contact with one or more alkaline substances.

In a particular aspect thereof, the manufacturing process defined herein obtains the oral dosage form.

In a further aspect thereof, the oral dosage form may be further defined in terms of its stability. Though an aqueous solution is avoided during the manufacturing of the oral dosage form the provided compositions still may have a water content on the same level as compositions produced by wet-granulation.

Very surprisingly, as demonstrated in example 10 that when batches produced by means of co-milling were compared with batches produced by means of wet-granulation and where all batches had a water content on the same level irrespective of the manufacturing process, there was a significant improvement in stability of the batches manufactured by co-milling. The mechanism of the improvement in stability is not understood in detail but without being limited to a specific theory it is contemplated that the impact of the granulation liquid in case of wet-granulation alters the crystal structure of the active drug substance. This alteration of crystal structure does not take place in the compositions produced by co-milling. By the present inventor it is therefore suggested, without being limited to this theory, that when contacting a drug substance, as demonstrated with lornoxicam, with a granulation liquid the drug substance, in this case lornoxicam partly dissolves for subsequently to solidify into a less stable form. This less stable form is likely to be an amorf form of the drug substance, e.g. of the active drug substance. From the promising stability results on lornoxicam it is concluded that this process will also be advantageous for other drug substances, such as other NSAID including thiazinecarboxamides.

The improved stability was demonstrated in a stability study, as described in example 10, where co-milled compositions were compared to a batch produced by means of wet-granulation. In this study the degradation product of lornoxicam, HN-10004 was chosen as the stability indicating parameter. In this instance the co-milling was performed by means of a ball mill with horizontally moving spheres. It is, however, firmly believed that the type of co-milling will not influence the stability as the decisive factor for the stability is whether or not the active drug substance is contacted with a granulation liquid. Other co-milling procedures as described herein are therefore also suitable.

At the starting point of the test period the water-content of the tablets were determined. The water-content of all compositions was on the same level, with a tendency of the batch produced by wet-granulation to be lower in water-content than the two other compositions. Until now it has been well-known that the stability of lornoxicam in combination with an alkaline substance is closely related to a low water content in the tablet.

In the stability study the stability was tested at several test points over a period of 6 months as appears from example 10. As the stability indicating parameter was in example 3010 chosen the decomposition product HN-10004. At all test points the amount of HN-10004 in the batch nos. 17110431 and 17110432 (co-milled) was lower than in batch no. 10225671 (wet-granulated).

At all the test points the stability of the two co-milled compositions are superior to a significant extent over the wet-granulated batch, despite the fact that the wet-granulated batch has a lower water-content.

At the starting point, the amount of HN-10004 is also lower in the co-milled product showing that decomposition takes place even at the time of production in the wet-granulated batches.

Very surprisingly the co-milling process thus leads to a product with a significantly improved stability irrespective of the higher water content.

The co-milling process was demonstrated with lornoxicam as a model substance, and it is most likely that this process will be suitable for other active drug substances. Such drug substances are typically weak acid where the alkaline substance will function as a dissolution aid, and the co-milling process is particularly suitable for active drug substances with stability problems.

In suitable embodiments of the invention, the oral dosage form further comprises a binder, which unlike a number of conventional manufacturing processes, has not been presented to aqueous solution during the manufacturing process or pre-treated with aqueous solution before use. Therefore, the binder is not present in swelled form, as may be determined under a microscope, e.g. a scanning electron microscope (SEM) with sufficient magnifying effect.

It is to be understood, that the manufacturing process and oral dosage forms of the invention may be characterised in terms of
type of therapeutically active substance
type of alkaline substance
mixing process and the resulting mixture of the active drug substance and alkaline substance
quick dissolution of the active drug substance in acidic solution
stability of the therapeutically active ingredient;

These aspects will be discussed in the following.

In some embodiments the manufacturing process and the oral dosage form of the invention may further be characterised in terms of the process of compressing tablets. E.g. for a 10 mm round standard concave tablet a force of minimum app. 4 kN is applied.

Results from the Experiments

Different equipment for co-milling can be implied in the process. Below is presented an overview of type of co-milling equipment, parameters and examples enclosed herein.

| Type of | | | | |
|---|---|---|---|---|
| co-milling | Specific type of co- | Force applied by the | | |
| equipment | milling equipment | equipment/parameters | Time of impact | Ex. |
| Roller | Minipactor ® from | High force | Short time | 6 |
| compaction | Gerteis Maschinen + | Compaction force: 8-12 kN/cm | such as less | |
| | Processengineering | Rpm: 2 | than 1 min. | |
| | AG. | Sieve size: 1.0-1.5 mm | | |
| | | Gab size: 2.5 mm | | |
| Ball mill, | Struers ball mill | Low force | Long time: | 11 |
| vertically | | 250-400 rpm | ½-24 hours | |
| moving spheres | | sieve: 700 μm mesh. | | |
| Ball mill with | Fritsch Pulverisette | Medium force | Medium time | 2 |
| horizontally | type 06.002.00 | | 5 to 30 min. | 3 |
| moving spheres | | | | 4 |
| | | | | 10 |
| Mechano fusion | AMS-LAB mechano | Medium force | Medium time | 5 |
| | fusion unit from | Time: 3-30 min | 5 to 30 min. | |
| | Hosokawa Alpine. | Rotor speed: | | |
| | | 1300-1500 | | |
| | | Temp: 20-45° C. | | |
| Milling alkaline, | — | — | | 7 |
| admixture | | | | 8 |
| Simple mixture | — | — | | 9 |

Examples 2, 3, 4 and 10 relate to co-milling provided by ball milling with horizontally moving spheres. The alkaline substance used in example 2 is trisodium phosphate. Two types of binder is used; HPC and VA 64 both combinations providing a fast dissolution of the resulting tablets. The co-milling is in this instance provided by a medium force and medium impact time. In example 3 the alkaline substance is sodium carbonate and the milling process is again ball milling with horizontally moving spheres. Similarly to example 2, two types of binder is used; HPC and VA 64 both combinations providing a fast dissolution of the resulting tablets. Finally in example 4 arginine is used as the alkaline substance providing tablets with a fast release that is on the same level as that obtained with trisodium phosphate. The stability of tablets provided by the procedure described in examples 2 and 3 were tested in a stability study, example 10. The tablets displayed excellent stability compared to tablets provided by known manufacturing methods.

Another principle of co-milling also providing a medium force and a medium impact time was investigated, Mechano fusion milling. In example 5 tablets are provided after co-milling with a AMS-LAB mechano fusion unit from Hosokawa Alpine. The tablets either contained trisodium phosphate, sodium carbonate, arginine or lysine as the alkaline substance. All compositions had a fast dissolution, the batch with lysine showed outstandingly fast dissolution results. Two compositions are based on trisodium phosphate with different mean particle sizes of the alkaline substance; 203 μm, 40 μm respectively. Though both the batch with the large as well as the batch with the small particle size gave acceptable results, dissolution was significantly faster in the batch with the smaller particle size.

To demonstrate that a low force combined with a long impact time can provide the co-milling effect, tablets were produced with lysine or trisodium phosphate as the alkaline substance in example 5. Furthermore the batches produced with a molar ratio lornoxicam:alkaline substance of 1:20 was compared with batches produced with a molar ratio of 1:40. All batches displayed fast dissolution and the molar ratio had no impact on the dissolution rate.

Finally, a high force combined with a short impact time was provided by roller compaction. In example 6 tablets were provided by means of Minipactor® from Gerteis Maschinen+Processengineering AG using the alkaline substances trisodium phosphate or lysine. Batches were produced, based on both a small and a large mean particle size of the alkaline substance. All batches showed fast dissolution and the particle size did not seem to have major impact with this co-milling method.

Alternatively the close contact provided by co-milling can be established by milling an alkaline substance in the equipment used for co-milling or other suitable equipment followed by a simple admixture or a blending and subsequent tabletting, where the tabletting provides a high force in a short impact time.

In example 7 lysine as the alkaline substance was milled in Hosokawa Alpine spiral jet mill to a mean particle size of 5 μm followed by compression of the mixture of ingredients into tablets. Two batches were produced that both showed a fast dissolution.

From the examples it is concluded that either a co-milling of the active drug substance together with the alkaline substance or a milling procedure of the alkaline substance followed by compression into tablets or alternatively a wet-granulation, as is described in the prior art, is needed to provide tablets with a fast dissolution. To demonstrate this statement, tablets were produced according to the prior teaching (e.g. 3P 3240729 or EP 1109534), namely the wet-granulation process but without the wetting step. This experiment is described in more detail in example 9 and as is seen in the example, the resulting tablets have an unacceptable slow dissolution.

Conclusively, the examples demonstrate that a fast dissolution is provided by co-milling of a therapeutically active drug substance having a solubility at room temperature of less than 0.1% w/v in 0.1N hydrochloric acid or which has a pKa of less than 5.5 together with an alkaline substance, optionally followed by admixture of other excipients and optionally followed by tabletting of the mixture of ingredients.

Alternatively, the alkaline substance can by milling according to the methods described herein for subsequently to be mixed with the active drug substance, optionally followed by admixture of other excipients for subsequently to be compressed into tablets.

Finally the fast dissolution can be provided by co-milling of the active drug substance together with an alkaline substance and optionally together with other excipients followed by optional admixture of even further other excipients and subsequently followed by compressing the mixture of ingredients into tablets.

### Therapeutically Active Ingredient

In principle any active ingredient characterised by having a poor solubility in acidic solution, may be processed by the above-mentioned manufacturing process in order to improve the dissolution in acidic solution and thus ensuring fast absorption of the drug in the upper gastrointestinal tract upon orally administering the resulting composition.

According to the present invention, the active ingredient is a therapeutically active compound with poor solubility in 0.1 N hydrochloric acid, such as a solubility in 0.1 N hydrochloric acid of less than 0.1% w/v. Alternatively defined, the therapeutically active compound has a pKa value of less than 5.5 in that such compounds are also known to dissolve poorly in the gastric fluid. Furthermore, the active ingredient may be defined as belonging to the group of NSAID's that are characterised by being weak acids. Examples on NSAID's are lornoxicam and naproxone.

A majority of the active drug substances mentioned are weak acids, i.e. substances which have a pKₐ value below about 5.5 such as, e.g., In a range of from about 3.0 to about 5.5 or in a range of from about 4.0 to about 5.0. In this connection it can be mentioned that the pKₐ value for lornoxicam is about 4.7, for naproxen about 4.2, for indometacin about 4.5, for ibuprofen about 5.2 and for acetylsalicylic acid about 3.5. Moreover, active drug substances like those mentioned above generally have a poor solubility in media having a pH below the pKₐ value. As an example the solubility of lornoxicam at a pH of 0.1 N HCl is less than about 1 mg/100 ml at room temperature. Active drug substances like acetylsalicylic acid, indometacin and naproxen are regarded as substances, which are practically insoluble in water and in 0.1 N HCl at room temperature.

The term “active drug substance” is in the present description and claims used synonymously with “therapeutically active substance”, “therapeutically active ingredient” and “therapeutically active compound”.

Likewise, the term “pharmaceutical composition” is in the present description and claims used synonymously with “pharmaceutical formulation”, “formulation” and “dosage form”.

Relevant examples of active drug substances suitable for use in compositions according to the invention are in general weak acidic substances such as, e.g., paracetamol and/or NSAID substances like
aminoarylcarboxylic acid derivatives like e.g. enfenamic acid, fiufenamlc acid, isonixin, meclofenamic acid, mefenamic acid, morniflumate, niflumic acid, and tolfenamic acid,
arylacetic acid derivatives like e.g. aceclofenac, acemetacin, amfenac, bromfenac, cimmetacin, diclofenac, etodolac, fentlazac, glucametacin, indomethacin, lonazolac, metlavinic acid, oxametacine, pirazolac, proglumetacin, sulindac, tiaramide, tolmetin, and zomepirac,
arylcarboxylic acids like e.g. ketorolac and tinoridine, arylpropionic acid derivatives like e.g. alminoprofen, bermoprofen, carprofen, dexibuprofen, fenbufen, fenoprofen, flunoxaprofen, flurbiprofen, ibuprofen, ibuproxam, ketoprofen, loxoprofen, naproxen, oxaprozin, pranoprofen, protizinic acid, and tiaprofenic acid,
pyrazoles like e.g. epirizole,
pyrazolones like e.g. benzpiperylon, mofebutazone, oxyphenbutazone, phenylbutazone, and ramifenazone,
salicylic acid derivatives like e.g. acetaminosalol, acetylsalicylic acid, benorylate, eterisalate, fendosal, imidazole salicylate, lysine acetylsalicylate, morpholine salicylate, parsalmide, salamidacetic acid and salsalate, thiazinecarboxamides like a.o. ampiroxicam, droxicam, lornoxicam, meloxicam, piroxicam, and tenoxicam,
others like bucillamine, bucolome, bumadizon, diferenpiramide, ditazol, emorfazone, nabumetone, nimesulide, proquazone, acrivastine and piroxicam (e.g. in the form of a betacyclodextrin complex), wherein the NSAID may be in the form of a pharmaceutically acceptable salt or a prodrug.

From a market point of view especially the following NSAID's are interesting: lornoxicam, diclofenac, nimesulide, ibuprofen, piroxicam, piroxicam (betacyclodextrin), naproxen, ketoprofen, tenoxicam, meloxicam, tolfenamic acid, bromazepam, aceclofenac, indometacin, nabumetone, acemetacin, morniflumate, meloxicam, flurbiprofen, tiaprofenic acid, proglumetacin, mefenamic; acid, fenbufen, etodolac, toifenamic acid, sulindac, phenylbutazone, fenoprofen, tolmetin, acetylsalicylic acid, dexibuprofen and pharmaceutically acceptable salts, complexes and/or prodrugs and mixtures thereof.

In particular the following NSAID's are interesting: piroxicam, meloxicam, ibuprofen, tolfenamic acid and bromazepam.

Other relevant active drug substances are COX-2 (COX is an abbreviation for cyclooxygenase) inhibitors like e.g. celecosib and flosulide.

At present, the most preferred drug substance is lornoxicam and pharmaceutically acceptable salts, complexes and/or prodrugs thereof, such as esters thereof. Lornoxicam may be present in a composition according to the invention as the sole drug substance or in combination with other drug substances such as opiolds or triptan's. Relevant examples of opioid substances are morphine, hydromorphone, codeine, oxycodone, hydrocodone, methadone, levorphanol, fentanyl, buprenorphine, butorphanol tartrate, dezocine, nalbuphine hydrochloride and meperidine. Relevant examples of triptan substances are metoclopramide, sumatriptan, rizatriptan, naratriptan, coimitriptan, eletriptan, almotriptan, zolmitriptan and frovatriptan.

In those cases where a quick release composition of the present invention includes an NSAID substance as the therapeutically active ingredient, the amount of the active drug substance corresponds to from 1 to about 1600 mg by weight. Alternatively, the dosage form may contain molar equivalent amounts of pharmaceutically acceptable salts thereof. The dosage form contains an appropriate amount to provide a substantially equivalent therapeutic effect.

In preferred embodiments of the invention, the active ingredient is lornoxicam. This drug is a weak acid (pKa less than 5.5) and has solubility in 0.1 N HCl less than 0.1% w/v and is prone to degradation in the presence of water. The tendency of degradation in presence of water is dependent of the presence of excipients, such as alkaline substances, and are in particular dependent on the manufacturing process as was demonstrated in the present specification.

The dissolution rate of an active drug substance may further be affected by the particle size of the drug substance. Accordingly, in interesting embodiments of the invention, the active drug substance may be further defined in terms of its particle size distribution. The particle size distribution can be determined by laser diffraction (e.g. using a Malvern Mastersizer 2000). The particle size distribution is calculated according to the Frauenhofer respective Mie theories. The sample is first dispersed in tenside solution as pre-treatment method. Then an aliquot of the pre-dispersion is transferred to a dispersion bath where further particle dispersion occurs while stirring and treating with ultrasound. This suspension is circulated through the measuring cell. During measurement the stirring remain active whereas the ultrasound is switched off.

Typically, the particle size distribution as determined by the above-mentioned laser diffraction of the active drug substance, is such that at least 90% by volume has a particle size below 100 μm, preferably below 75 μm, more preferably below 50 μm, such as about 40 μm. In some embodiments the particle size distribution is such that at least 95% by volume has a particle size below 32 μm, such as below 20 μm or most preferably below 10 μm. In other embodiments the particle size distribution is such that at least 80% by volume has a particle size below 10 μm.

However, as may be understood, the use of particle sizes in the lower range may not be practically acceptable. Thus, in still more interesting embodiments of the invention, the active drug substance has a particle size distribution, as determined by laser diffraction, wherein at least 90% by volume of the active drug substance has a particle size above 0.1 In some embodiments the mean particle size, D(v;0.5) (μm), is used. Typically, the mean particle size relates to an excipient, such as the mean particle size of an alkaline substance. By the mean particle size is understood a particle distribution determined by laser diffraction as described above, wherein the distribution of the particles are such that 50% of the particles are above and 50% of the particles are below the mean particle size, and wherein the distribution is determined by volume.

### Alkaline Substance

As stated, the oral dosage form of the invention should further comprise an alkaline substance. It is considered important that the alkaline substance is in physical contact with the active drug substance, such as lornoxicam. It is thought that the alkaline substance enables a microenvironment around the active drug substance so as to aid the dissolution of the active drug substance in acidic solutions when the composition is being exposed to acidic solution or water.

Typically the molar ratio between the active drug substance and the alkaline substance ranges between 1:100 and 1:1, preferably, the said molar ratio is 1:80, 1:60, 1:40 or 1:30, most preferably 1:20. In still other embodiments the molar ratio of the active drug substance and the alkaline substance is 1:10. The ratio of 1:10 is especially interesting in the embodiment wherein the alkaline substance is an amino acid or a derivative thereof, e.g. lysine, histidine or arginine or a derivative thereof.

As used herein, the term an “alkaline substance” is meant to include substances that provide an alkaline pH in the range of 8-14, preferably 8-13, when being dissolved in water at room temperature in an amount of about 10 mg/ml.

Accordingly, the term “alkaline substance” includes the corresponding base of an organic or an inorganic acid, such as provided in the form of a pharmaceutically acceptable salt of an organic or inorganic acid and mixtures thereof, organic amines and some amino acids or derivatives thereof. Typically, the organic or inorganic acid from where the corresponding base derives has a pKa in the range of 4-14.

Relevant alkaline substances are listed in table 1.

TABLE 1
List of alkaline substances

| Substance | Examples | Structure | pKa* |
|---|---|---|---|
| Salts of carbonic acid | Di-sodium carbonate = | Na₂CO₃ | 10.3 |
| (carbonates and | Sodium carbonate | | |
| hydrogencarbonates) and | Sodium hydrogen- | NaHCO₃ | 6.4 |
| phosphoric acid | carbonate = | | |
| (phosphates and mono | Sodium bicarbonate | | |
| hydrogenphosphates). | Tri-sodium phosphate | Na₃PO₄ | 12.4 |
| Soluble salts with pKa- | Di-sodium hydrogen- | Na₂HPO₄ | 7.2 |
| values from 4 to 11 | phosphate | | |
| Salts of organic acids | Sodium acetate | CH₃COONa | 4.8 |
| with pKa-values from 4 to 11 | Sodium citrate | C₆H₅O₇Na₃ | 6.4 |
| | Sodium maleate | C₄H₂O₄Na₂ | 6.2 |
| | Sodium fumerate | C₄H₂O₄Na₂ | 4.4 |
| | | (trans) | |
| Organic amines | Hydroxylamine | NH₂OH | 6 |
| | Diethyl amine | (CH₃CH₂)₂NH | 11 |
| | Triethyl amine | (CH₃CH₂)₃N | 10.8 |
| | Hydrazine | NH₂NH₂ | 8 |
| | Codeine | C₁₈H₂₁NO₃ | 8.2 |
| | | | (pH in saturated |
| | | | solution ≈9.8) |
| Amino acids with pKa₃- | Lysine | C₆H₁₄O₂N₂ | pKa₁: 2.2 |
| values from 8 to 14 | | | pKa₂: 8.9 |
| | | | pKa₃: 10.3 |
| | Arginine | C₆H₁₄N₄O₂ | pKa₁: 2.2 |
| | | | pKa₂: 9.1 |
| | | | pKa₃: 13.2 |
| | | | pH≈11.4 |
| | | | (100 g/L H₂O) |
| | Histidine | C₆H₉O₂N₃ | pKa₁: 1.8 |
| | | | pKa₂: 6.0 |
| | | | pKa₃: 9.0 |
| | | | pH≈7.7 |
| | | | (10 g/L H₂O) |

*The pKa-values in this table are approximate values and refer to the pKa of the acid.

In one embodiment of the present invention, the alkaline substance is a salt of an organic or inorganic acid or a mixture thereof, the organic or inorganic acid has a pKa in the range of 4-14, preferably in the range of 6 to 13.5, even more preferable in the range of 7-13, most preferably in the range of 8-13, such as 8.5-13, such as 9-13, such as 9-12.5.

In some embodiments of the present invention the alkaline substance is a salt of an inorganic acid selected from carbonic acid or phosphoric acid, such as hydrogencarbonic acid, dihydrogenic phosphoric acid and hydrogenic phosphoric acid.

That is to say that the salt has as the anion, an anion selected from carbonate, phosphate, and hydrogenphosphate group and as the kation, an earth metal selected from sodium, potassium, calcium, magnesium and the like, e.g. a salt containing an anion selected from CO₃²⁻, HPO₄²⁻, PO₄³⁻ and a kation selected from Na⁺, K⁺, Ca²⁺, Mg²⁺.

Typically, the salt of an inorganic acid is selected from di-sodium carbonate, di-sodium hydrogenphosphate and tri-sodium phosphate. Typically, hydrogencarbonate salts are not so feasible because of the effervescent effect unless this is an object.

In yet some embodiments of the present invention the alkaline substance is a salt of an organic acid, the organic acid being selected from citric acid, maleric acid or acetic acid. That is to say wherein the salt has an anion selected from acetate, hydrogencitrate, citrate, hydrogenmaleate or maleate. Typically, the salt of an organic acid is sodium acetate, trisodium citrate, disodiumhydrogencitrate or disodium maleate, and preferably trisodium citrate.

In still another embodiment of the present invention, the alkaline substance Is an organic amine selected from hydroxylamine, diethyl amine, triethyl amine or hydrazine.

In still another embodiment of the present invention the alkaline substance is an amino acid such as histidine, lysine or arginlne.

It is further contemplated that the alkaline substance should be soluble in water, such as at least to comply with the category of being sparingly soluble in water: 1 part of alkaline substance can be dissolved in a maximum of 100 parts of water. Preferably, the alkaline substance should be soluble in water: 1 part of alkaline substance is dissolved in a maximum of 30 parts of water.

Furthermore, it should be understood that the alkaline substance should be provided in solid form, such as in the form of a powder, granulate or the like.

### Process

As mentioned, the invented process includes a first step of mixing the active drug substance and the alkaline substance utilising intensive mixing by a mechanical process.

Importantly, the said mixing step has one object, namely to ensure the close physical contact between the active drug substance and the alkaline agent so as to achieve the desired microenvironment. To ensure the close physical contact, it may be appropriate not to add further excipients and to use the active drug substance and the alkaline substance in molar ratios ranging between 1:100 and 1:1. Preferably, the said molar ratio is 1:80, 1:60, 1:40 or 1:30, most preferably 1:20.

Typically, it is not an object of the said mixing procedure to affect the particle size of the active drug substance. In some instances, it may be expected that the intensive mixing may decrease the particle size of the alkaline substance, when being applied in particle sizes above 100 μm.

Importantly, the mixing should be undertaken under conditions excluding the addition of liquid such as aqueous liquids, water, mixtures of organic solvents and water, so as to provide conditions potentially reducing the degradation of the active drug substance. Thus, any step of conventional wet-granulation is excluded from the process.

The mixing is carried out by a mechanical process, which transfers energy to the mixture of active drug substance and alkaline agent so as to bring the active drug substance and the alkaline substance in close physical contact, much closer than expected with conventional mixing under formation of a particulate matter. The resulting particulate matter comprises the active drug substance and alkaline substance substantially homogeneously mixed within each other, but not molecularly dispersed within each other. Thus, it should be understood that the resulting particulate matter contains each of the constituents (alkaline substance and drug substance) as separate particles.

In principle any mechanical process resulting in the particulate matter as defined herein can be applied. Generally, the mechanical process requires intensive mixing such as the one provided by co-milling. By the term “co-milling” is meant a highly intensive mechanical mixing of two or more substances which bring these two substances in close physical contact with each other, closer than by using a conventional mixing procedure such as tumble mixing procedure.

The term “co-milling” as used herein is also meant to include any process achieving the same particulate matter as that obtained by co-milling, for example the mixing provided under dry granulation e.g. roller compaction as discussed herein.

The co-milling process is preferably applied only to the active drug substance and the alkaline substance, but lower amounts of other ingredients may be added in the case that the fast dissolution is achieved. In the embodiments where the alkaline substance has a tendency of sticking, which was observed in relation with using some amino acids as the alkaline substance, it is advantageous to add lower amounts of other ingredients such as calcium monohydrogen phosphate, anhydrous (CaHPO₃), trisodium phosphate (Na₃PO₄), magnesium aluminium silicate, magnesium oxide, calcium carbonate (CaCO₃), calcium sulphate dihydrate (CaSO₄, 2H₇O), sorbitol or talc. In a preferred embodiment, the co-milling is carried out on a mixture consisting essentially of the active drug substance and the alkaline substance.

In one embodiment the milling is performed only on the alkaline substance and the milling is provided by the same methods as the co-milling or other suitable equipment. The milling is typically resulting in a mean particle size of the alkaline substance in the range of 1 to 400 μm, such as 1 to 300 μm, such as 5 to 200 μm. Following the milling of the alkaline substance this substance is admixed with the active drug substance having a particle size distribution of at least 95% by volume has a particle size below 32 μm, such as below 20 μm or most preferably below 10 μm. In other embodiments the particle size distribution is such that at least 80% by volume has a particle size below 10 μm. By admixing is Intended a mixing procedure where the procedure does not imply a significant force on the mixture but only has the aim of mixing the components. The admixing is followed by compression into tablets, which compression has the effect of bringing the active dug substance and the alkaline substance into such a close contact that is otherwise provided by co-milling.

In an interesting embodiment of the invention, the mechanical process is co-milling. Co-milling can be achieved by using standard milling equipment, such as Hammer Mill (e.g. Fitz Mill, supplied by Fltz Patrick). The co-milling process may also be carried out using a Ball Mill (e.g. Fritz Pulverizette), which is a ball mill with horizontally moving spheres. Another principle is a ball mill having vertically moving spheres, such as a Struers ball mill also available at Hosokawa. A mechano fusion equipment (as supplied by Hosokawa) or a Micros Ring Mill. Finally, can a roller compactor provide co-milling, e.g. Minipactor® from Gene's Maschinen+Processengineering AG.

By the term “co-milling” is thus understood a process that results in creating a close physical contact between lornoxicam and an alkaline substance. This contact can be created by use of a relatively high force combined with a relatively short period of impact as when using roller compaction. When using roller compaction the compaction force is typically in the range of 6-14 kN/cm with an impact time of less than 1 minute. Compaction of tablets also provides a relatively high force combined with a relatively short period of impact. Tabletting typically provides a force of 4 kN or more for a standard concave round 10 mm tablet and an impact time of less than 1 minute. Alternatively using a relatively low force combined with a longer time of impact can provide the same degree of co-milling. As an example, ball mill with vertically moving spheres provides a low force, in the latter case a considerably longer time of impact is required. The use of an intermediate amount of time of impact is also possible when a medium force is provided by mechano fusion or ball milling with horizontally moving spheres. If the alkaline substance has a small particle size, the physical contact can be created by a simple mixing and subsequent compression into tablets. Said small particle size can be obtained by milling.

### Ball Milling

Co-milling performed by ball milling can be divided into ball milling performed with horizontally moving spheres or with vertically moving spheres. Utilising ball milling performed with horizontally moving spheres provides a medium force intensity and thus needs a medium time of impact, such as 5 to 30 minutes. An example of an equipment suitable for performing ball milling with horizontally moving spheres is Fritz Pulverizette. Ball milling with vertically moving spheres provides a low force and therefore requires a long impact time. An example of equipment suitable for performing ball milling with vertically moving spheres is Struers ball mill.

### Mechano Fusion

The basic operation principle is to circulate a powder by a rotor while receiving a strong force when meeting a press head. This procedure is repeated at high speed thereby forming the powder into a particulate matter. An example of equipment is AMS-LAB mechano fusion unit from Hosokawa Alpine.

### Roller Compaction

The working principle of roller compaction is to press powder between 2 counter rotating rollers to make a solid sheet which is subsequently crushed in a sieve to form a particulate matter. In this particulate matter a close mechanical contact between the powder has been obtained. An example of equipment is Minipactor® from Gerteis Maschinen+Processengineering AG.

By the term “micronised” as used herein is intended particles having a mean particle size below app. 5 μm resulting from a milling process.

By the term “rpm” is to be understood “rotations pr. minute”. The term is typically used to describe the number of revolution of a moveable part of equipment such as the blade of a mixing equipment or the paddle of a dissolution equipment.

By the term “sieve size” is to be understood the diameter of the mesh of a sieve.

By the term “RH” is to be understood the “relative humidity”, typically describing the amount of water vapour present in the air at a defined temperature.

In principle, the resulting mixture of the active drug substance and the alkaline substance (the co-milled mixture) of the invention can be used directly for making orally administrable dosage forms. That is to say, without the addition of further pharmaceutically acceptable excipients.

However, in some embodiments of the invention the process comprises a second step comprising admixing of one or more pharmaceutically acceptable excipients to said particulate matter using conventional mixing, such as by tumble mixing. Thus, an oral dosage form of the invention may comprise one or more further pharmaceutically acceptable excipients, such as filler (diluent), binder, disintegrant, glidant, colours and so forth. Often the dosage form will comprise a filler, or a binder, or a disintegrant, or a glidant, or a colour or a combination of one or more of the excipients.

For example, the further pharmaceutically acceptable excipient may be selected with the object of providing an oral dosage form in the form of a tablet, a pill, a capsule, a sachet or the like.

Accordingly, in one preferred embodiment of the invention, the process further comprises the step of compressing the particulate matter under tabletting conditions so as to achieve a tablet. Compressing a powder into a particulate matter improves the flowability by tabletting, which may further improve the dissolution of the active substance.

In one embodiment the further pharmaceutically acceptable excipients is a binder, preferably a hydrophilic binder selected from cellulose derivatives, saccharine or povidone. Typically, any binder can be applied as long as the resulting tablet has a disintegration time in water at 37° C. of less than 30 minutes, preferably less than 15 minutes, more preferably less than 5 minutes.

Thus, a further aspect of the invention relates to a pharmaceutical composition for oral administration comprising:
an active drug substance which has a solubility at room temperature less than 0.1% w/v in 0.1 M hydrochloric acid or has a pKₐ value less than 5.5;
one or more alkaline substances; and
a binder in the form of a hydrophilic polymer such as cellulose derivatives, saccharine or povidone

### Dissolution Properties

In contrast to what was previously known, it was shown evident that a fast dissolution of an active drug substance can be obtained by utilising a process excluding the addition of liquid, such as aqueous liquid, to a particulate matter that comprises the active drug substance and the alkaline substance.

Therefore, in one embodiment of the invention, the resulting particulate matter or the mixture of ingredients of the active drug substance and alkaline substance, optionally in admixture with further excipients, has an in vitro dissolution profile, when being subjected to a dissolution test method using 1300 ml of 0.1 N HCl or 0.07N HCl equilibrated at 37° C. as the dissolution medium and USP paddle dissolution apparatus II applied with a stirring rate of 50 rpm, characterised in that at least 50% w/w of the active substance is present on dissolved form in the dissolution medium at the time point of 20 minutes after start of the dissolution testing.

In another embodiment a dissolution test method was applied using a stirring rate of 150 rpm but otherwise maintaining the same parameters as described above is applied. The in vitro dissolution profile of the pharmaceutical composition is characterised in that at least 50% w/w of the active substance is present on dissolved form in the dissolution medium at the time point of 20 minutes after start of the dissolution testing.

Preferably, the resulting particulate matter or the mixture of ingredients has an in vitro dissolution profile, characterised in that at least 55% w/w, such as at least 60% w/w, at least 65% w/w, at least 70% w/w, at least 75% w/w, or at least 80% w/w of the active substance is present on dissolved form in the dissolution medium at the time point of 20 minutes after start of the dissolution testing.

As stated, further pharmaceutically acceptable excipients may be added to the particulate matter, for instance with the object to further improve the dissolution rate.

In preferred embodiments of the invention, the particulate matter is compressed into a tablet. The present inventor provides herein evidence that the further step of compressing the particulate matter provides an even faster dissolution of lornoxicam in 0.1 N HCl.

Thus, in a further embodiment, the resulting tablet has an in vitro dissolution profile, when being subjected to dissolution test method using 0.1 N HCl equilibrated at 37° C. as the dissolution medium and USP paddle dissolution apparatus applied with a stirring rate of 50 rpm as the equipment, characterised in that at least 75% w/w of the active substance is present on dissolved form in the dissolution medium at the time point of 20 minutes after start of the dissolution testing.

Preferably the resulting tablet has an in vitro dissolution profile, characterised in that at least 80% w/w, such as at least 85% w/w, at least 90% w/w, at least 95% w/w of the active substance is present on dissolved form in the dissolution medium at the time point of 20 minutes after start of the dissolution testing.

### Stability

The oral dosage form of the invention has a shelf life at least as good as the one processed with the addition of aqueous liquids. During the development phase it has surprisingly become evident that the stability of the co-milled compositions are significantly better than conventionally produced compositions, despite the water content being on the same level.

Therefore, a particular embodiment of the invention relates to an oral dosage form comprising lornoxicam in physical contact with an alkaline substance, wherein the oral dosage form is chemically stable with respect to the lornoxicam, such that at least 85% by weight of the lornoxicam is present in the oral dosage after at least 3 months of storage at 25° C. and 60% RH in darkness. The storage may be performed in closed containers, blister packaging material, or in open containers. Preferably, at least 85% w/w, more preferably 90% w/w, even more preferably at least 95% w/w, most preferably at least 98% w/w of the lornoxicam is present in said composition or dosage unit after at least 6 months, preferably more than 12 months, even more preferably more than 24 months and most preferably more than 36 months of storage at the above-mentioned conditions.

Another way to define the stability aspect of the invention relates to the concentration of degradation products or impurities in the said oral dosage form of lornoxicam. The concentration of degradation products present in the oral dosage is determined after at least 3 months, such as at 3 months, of storage at 25° C. and 60% RH in darkness. The storage may be performed in closed containers, blister packaging material, or in open containers. Preferably, the total sum of degradation products in the oral dosage form amounts to less than 15% of the initial amount of lornoxicam, more preferably less than 10%, even more preferably less than 5° k, most preferably less than 2% w/w.

This allows for a shelf life of more than 6 months, preferably more than 12 months, even more preferably for more than 24 months and most preferably more than 36 months.

There is reason to believe that when formulating a composition according to the invention using other active drug substances, such as NSAIDs, e.g. thiazinecarboxamides that these other active drug substances will also meet the above described stability requirements.

### Further Excipients

As noted above, the oral dosage form of the invention may comprise a number of additional pharmaceutically acceptable excipients other than the alkaline substance, such as solvents, surfactants, binders, fillers, disintegrants, coatings, diluents, glidants, stabilisers, lubricants, artificial sweeteners, flavouring agents, buffering agents or colorants.

Disintegrating agents may be incorporated such as e.g. alginic acid-alginates, carboxymethylcellulose calcium, carboxymethylcellulose sodium, crospovidone, hydroxypropylcellulose, hydroxypropylmethylcellulose (HPMC), cellulose derivatives such as low-substituted hydroxypropylcellulose (e.g LH 11, LH 20, LH 21, LH 22, LH 30, LH 31, LH 32 available from Shin-Etsu Chemical Co.) and microcrystalline cellulose, polacrilin potassium or sodium, polyacrylic acid, polycarbofil, polyethylene glycol, polyvinylacetate, polyvinylpyrrolidone (e.g. Polyvidon® CL, Polyvidon® CL-M, Kollidon® CL, Polyplasdone® XL, Polyplasdone® XL-10); sodium carboxymethyl starch (e.g. Primogel® and Explotab®), sodium croscarmellose (i.e. cross-linked carboxymethylcellulose sodium salt; e.g. Ac-Di-Sol®), sodium starch glycolate, starches (e.g potato starch, maize starch, rice starch), pre-gelatinised starch.

Those skilled in the art will appreciate that it is desirable for compressible tablets to disintegrate within 30 minutes, more desirable within 15 min, most desirable within 5 min; therefore, the disintegrant used preferably results in the disintegration of the tablet within 30 minutes, more preferable within 15 min, most preferable within 5 min.

Fillers/diluents/binders may be incorporated such as e.g. dextrins, maltodextrins (e.g. Lodex® 5 and Lodex® 10), dextrose, fructose, glucose, inositol, erythritol, isomalt, lactitol, lactose (e.g., spray-dried lactose, α-lactose, β-lactose, Tabletose®, various grades of Pharmatose®, Microtose or Fast-Floc®), maltitol, maltose, mannitol, sorbitol, sucrose, tagatose, trehalose, xylitol, low-substituted hydroxypropylcellulose (e.g LH 11, LH 20, LH 21, LH 22, LH 30, LH 31, LH 32 available from Shin-Etsu Chemical Co.), microcrystalline cellulose (e.g., various grades of Avicel®, such as Avicel® PH101, Avicel® PH102 or Avicel® PH105, Elcema® P100, Emcocel®, Vivacel®, Ming Tal® and Solka-Floc®), starches or modified starches (e.g potato starch, maize starch, rice starch, pre-gelatinised starch), polyvinylpyrrolidone, polyvinylpyrrolidone/vinyl acetate copolymer, agar (e.g. sodium alginate), calcium hydrogen phosphate, calcium phosphate (e.g. basic calcium phosphate, calcium hydrogen phosphate), calcium sulphate, carboxyalkylcellu lose, dextrates, dibasic calcium phosphate, gelatine, gummi arabicum, hydroxypropyl cellulose, hydroxypropylmethylcellulose, magnesium carbonate, magnesium chloride, methylcellulose, polyethylene glycol, polyethylene oxide, polysaccharides e.g. dextran, soy polysaccharide, sodium carbonate, sodium chloride, sodium phosphate.

Glidants and lubricants may be incorporated such as stearic acid, metallic stearates, talc, waxes and glycerides with high melting temperatures, hydrogenated vegetabable oils, colloidal silica, sodium stearyl fumarate, polyethylenglycols and alkyl sulphates.

Suitable lubricants include talc, magnesium stearate, calcium stearate, stearic acid, hydrogenated vegetable oils and the like. Preferably, magnesium stearate is used.

Surfactants may be incorporated such as non-ionic (e.g., polysorbate 20, polysorbate 21, polysorbate 40, polysorbate 60, polysorbate 61, polysorbate 65, polysorbate 80, polysorbate 81, polysorbate 85, polysorbate 120, sorbitane monoisostearate, sorbitanmonolaurate, sorbitan monopalmitate, sorbitan monostearate, sorbitan monooleate, sorbitan sesquioleate, sorbitan trioleate, glyceryl monooleate and polyvinylalkohol), anionic (e.g., docusate sodium and sodium lauryl sulphate) and cationic (e.g., benzalkonium chloride, benzethonium chloride and cetrimide) or mixtures thereof. Other appropriate pharmaceutically acceptable excipients may include colorants, flavouring agents, and buffering agents.

A film coating may also be applied on a composition according to the invention provided that the coating does not substantially retard the release of the active drug substance from the composition, but only to increase the swallowability, appearance, stability or in order to minimize any dusty problems.

Film coatings may be applied such as e.g. as hydroxypropylmethylcellulose (HPMC) (e.g. HPMC E5, HPMC E15), hydroxyethylcellulose, hydroxypropylcellulose, polydextrose and maitodextrin, Sepifilm™ and Sepifilrrt™ LP available from Seppic S. A., Pharmacoat® available from Shin-Etsu Chemical Co.

Film additives may be incorporated such as e.g. acetylated monoglyceride, acetyltributyl, acetyltributyl citrate, acetyltrlethyl citrate, benzyl benzoate, calcium stearate, castor oil, cetanol, chlorebutanol, colloidal silica dioxide, dibutyl phthalate, dibutyl sebacate, diethyl oxalate, diethyl malate, diethyl maleate, diethyl malonate, diethyl fumarate, diethyl phthalate, diethyl sebacate, diethyl succinate, dimethylphthalate, dioctyl phthalate, glycerin, glyceroltributyrate, glyceroltriacetate, glyceryl behanate, glyceryl monostearate, hydrogenated vegetable oil, lecithin, leucine, magnesium silicate, magnesium stearate, polyethylene glycol, propylene, glycol, polysorbate, silicone, stearic acid, talc, titanium dioxide, triacetin, tributyl citrate, triethyl citrate, zinc stearate, wax.

### Definitions of Selected Terms Used Herein

The term “shelf-life” is intended to mean the period of time, wherein the therapeutically active substances in a composition is stable at ambient conditions, e.g. 25° C. and 60% RH (relative humidity), such that at least 90%, preferably 95%, more preferably 98% of the initial amount of said substances is still present in the composition within the specified shelf-life.

The terms “quick release”, “fast release” or “enhanced release” in the present context refer to a modified release composition of which the release of the active drug substance and its subsequent absorption are fast. More specifically, the terms “quick release”, “fast release” or “enhanced release” mean that for a composition—when subjected to a dissolution method as described above wherein at least about 50% w/w of the active substance is dissolved within the first 20 min of the test.

The term “formulated” is intended to relate to the selection of excipients, carriers, vehicles, solvents, co-solvents, preservatives, colouring agents, flavouring agents and so forth in the preparation of a medicament using said composition. The term “formulated” is furthermore intended to relate to the selection of the device for delivery of the composition or selection of containment device for administration or storing of the composition.

In the present context, the term “pharmaceutically acceptable excipient” is intended to denote any material, which is inert in the sense that it substantially does not have any therapeutic and/or prophylactic effect per se. A pharmaceutically acceptable excipient may be added to the active drug substance with the purpose of making it possible to obtain a pharmaceutical formulation, which has acceptable technical properties.

The terms “NSAID's” or “NSAID substances” are used herein to designate a group of drugs that belongs to non-steroid anti-inflammatory drug substances and pharmaceutically acceptable salts, prodrugs and/or complexes thereof as well as mixtures thereof.

The terms “opioids” or “opioid substances” are used herein to designate a group of substances, pharmaceutically acceptable salts, prodrugs and/or complexes thereof as well as mixtures thereof that are used in the management of moderate to severe pain because of their effectiveness, ease of titration, and favourable risk-to-benefit ratio. Opioids produce analgesia by binding to specific receptors both within and outside the CNS.

The terms “triptans” or triptane substances” are used herein to designate a group of drug substances ad pharmaceutically acceptable salts, prodrugs and/or complexes thereof as well as mixtures thereof that act as agonists for 5-hydroxytryptamine (5-HT) receptors. The triptans are often very effective in relieving migraine but do not prevent future attacks or lessen their frequency.

### Further Embodiments

In one aspect a pharmaceutical composition obtainable by the process as defined in the description, where the pharmaceutical composition in one embodiment comprises:
an NSAID; and
an amino acid or a derivative thereof.

In another embodiment
the active drug substance is ampiroxicam, droxicam, lornoxicam, meloxicam, piroxicam, tenoxicam, bromazepam, ibuprofen, tolfenamic acid or dexibuprofen or a pharmaceutically acceptable salt or prodrug thereof;
the alkaline substance is histidine, lysine or arginine.

In another embodiment
the active drug substance is lornoxicam or a pharmaceutically acceptable salt or prodrug thereof;
the alkaline substance is histidine, lysine or arginine.

In another embodiment
the active drug substance is lornoxicam;
the alkaline substance is histidine, lysine or arginine.

In a Further Aspect

The pharmaceutical composition for oral administration comprises:
an NSAID or a pharmaceutically acceptable salt or prodrug thereof;
one or more alkaline substances selected from a salt containing an anion selected from CO₃²⁻, HPO₄²⁻, PO₄³⁻ and a kation selected from Na⁺ and K⁺;
   and a binder in the form of a hydrophilic polymer.

In a Still Further Aspect

A pharmaceutical composition for oral administration comprises:
an NSAID or a pharmaceutically acceptable salt or prodrug thereof;
one or more amino acids or a derivative thereof.

In one embodiment of this aspect the amino acid is histidine, lysine or arginine.

In one embodiment of both latter aspects, the NSAID is ampiroxlcam, droxicam, lornoxicam, meloxicam, piroxicam, tenoxicam, Ibuprofen or dexibuprofen or a pharmaceutically acceptable salt or prodrug thereof.

In one embodiment of both latter aspects, the NSAID is lornoxicam or a pharmaceutically acceptable salt or prodrug thereof.

In one embodiment of both latter aspects, the NSAID is lornoxicam.

In one embodiment of both latter aspects, the composition has an in vitro dissolution profile, when being subjected to dissolution test method using 0.1 N HCl equilibrated at 37° C. as the dissolution medium and USP paddle dissolution apparatus applied with a stirring rate of 50 rpm as the equipment, characterised in that at least 50% w/w of the active substance is present on dissolved form in the dissolution medium at the time point of 20 minutes after start of the dissolution testing.

### EXAMPLES

The following non-limiting examples are meant to illustrate the present invention.

### Examples

### Example 1

### Dissolution of the Resulting Particulate Matter and Oral Dosage Form

Dissolution Method for Testing the Release of Lornoxicam from a Tablet

### Dissolution Method

The following dissolution method alms at simulating the conditions in the stomach:
Apparatus: USP Dissolution Apparatus 2 equipped with paddles (as mentioned in USP 27 <711>)
Filters: Whatman GF/F glasfiber filters
Dissolution medium: 1300 ml of 0.1 N HCl with 2 g/l of sodium chloride added (examples 2 and 3) or 1300 ml of 0.07 N HCl with 2 g/l of sodium chloride added (examples 4 to 11)
Stirring rate: 50 rpm (examples 2 and 3) or 150 rpm (examples 4 to 11).
Temperature: 37° C.±0.5° C.
Sampling: Samples are taken every 5 minutes for a period of at least 60 minutes.
Quantification: The concentration of lornoxicam is determined in each sample using UV/Vis Spectrophotometer equipped with 10 mm cuvette and detection wavelength of 378 nm. E_{1 cm}^{1%}:587.0.
   Each sample was tested with n=3.

### Example 2

### The Effect of Co-Milling of Lornoxicam and Trisodium Phosphate on the Dissolution of Lornoxicam in Acid Solution

In the present example the co-milling was performed with a ball-mill having horizontally moving spheres.

Ingredients:

| | | | |
|---|---|---|---|
| 1. | Lornoxicam | 8 | mg/tablet |
| 2. | Trisodium phosphate (Na₃PO₄) | 78 | mg/tablet |
| 3. | Cellulose, microcrystalline | 96 | mg/tablet |
| 4. | Calcium monohydrogen phosphate, anhydrous | 110.4 | mg/tablet |
| 5. | Low substituted hydroxypropylcellulose | 48 | mg/tablet |
| 6. | Binder (either a or b) | 16 | mg tablet |
| | a) Hydroxypropylcellulose (HPC-L-fine) | | |
| | b) Vinylpyrrolidon-Vinylacetate | | |
| 7. | Calcium stearate | 1.6 | mg/tablet |
| | Total core mass: | 358 | mg |

Batch size: approx. 750 g

The amount of lornoxicam and trisodium phosphate was in the molar relationship of 1:20.

Lornoxicam (1) and trisodium phosphate (2) were co-milled for 5 minutes using a Fritsch Pulverisette (type 06.002.00; a ball mill with horizontally moving spheres). To the co-milled mixture of 1 and 2 was admixed further ingredients (3), (4), (5), (6) and (7) using an Erweka tumble mixer at 25 rpm for 5 min.

The resulting mixture (“mixture of ingredients 1-7”) was compressed into tablets using a Korsch 106 tabletting machine and 10.0 mm round standard concave punches.

Dissolution testing according to the method of Example 1 were conducted on the “mixture of ingredients 1-7” as well as on the resulting tablets.

TABLE 2
Dissolution results for both the “mixture of ingredients 1-7” and
corresponding tablets

| | | | % (w/w) dissolved |
|---|---|---|---|
| | | % (w/w) dissolved | lornoxicam at 20 min |
| Type of | | lornoxicam at | (n = 3); corrected |
| binder | Type of product | 20 min (n = 3)* | values |
| HPC | Powder mixture | 81 | 51 |
| | “mixture of | | |
| | ingredients 1-7” | | |
| | Tablet | 96 | 88 |
| VA 64 | Powder mixture | 77 | 54 |
| | “mixture of | | |
| | ingredients 1-7” | | |
| | Tablet | 92 | 82 |

*At a later stage in the development process the dissolution method proved to be misleading by giving too high values. The corrected values are listed in the last column.

As can be derived from Table 2, fast dissolution of lornoxicam in an acidic solution is achieved upon applying co-milling of lornoxicam and trisodium phosphate in that more than 50% of the lornoxicam is dissolved from the “mixture of ingredients 1-7” at the time point of 20 minutes after starting the dissolution testing. Furthermore, it can be concluded that the process of compressing the “mixture of ingredients 1-7” into tablets results in even higher dissolution of lornoxicam at 20 minutes after starting the dissolution testing. It is our theory that the compacting process which may strengthen the contact between lornoxicam and trisodium phosphate causes this.

### Example 3

### The Effect of Co-Milling of Lornoxicam and Sodium Carbonate on the Dissolution of Lornoxicam in an Acidic Solution

In the Present Example the Co-Milling was Performed with a Ball-Mill Having Horizontally Moving Spheres.

Ingredients:

| | | |
|---|---|---|
| 1. | Lornoxicam | 8 mg/tablet |
| 2. | Sodium carbonate decahydrate | 136.2 mg/tablet |
| | (Na₂CO₃, 10 H₂O)) | |
| 3. | Cellulose, microcrystalline | 96 mg/tablet |
| 4. | Calcium monohydrogen phosphate, anhydrous | 110.4 mg/tablet |
| 5. | Low substituted hydroxypropylcellulose | 48 mg/tablet |
| 6. | Binder (either a or b) | 16 mg tablet |
| | a. Hydroxypropylcellulose (HPC-L-fine) | |
| | b. Vinylpyrrolidon-Vinylacetate | |
| 7. | Calcium stearate | 1.6 mg/tablet |
| | Total core mass: | 416.2 mg |

Batch size was: Approx. 750 g

The amount of lornoxicam and sodium carbonate decahydrate is in a molar relationship of 1:20.

The “mixture of ingredients 1-7” as well as the corresponding tablets were made as described in example 2 and dissolution testing was carried out according to example 1.

The following dissolution results were obtained:

TABLE 3
Dissolution results for both the “mixture of ingredients 1-7” and
the corresponding tablets

| | | | % (w/w) dissolved |
|---|---|---|---|
| | | % (w/w) dissolved | lornoxicam at 20 min |
| Type of | | lornoxicam at | (n = 3); corrected |
| binder | Type of product | 20 min (n = 3)* | values |
| HPC | Powder mixture | 83 | 49 |
| | “mixture of | | |
| | ingredients 1-7” | | |
| | Tablet | 89 | 68 |
| VA 64 | Powder mixture | 75 | 51 |
| | “mixture of | | |
| | ingredients 1-7” | | |
| | Tablet | 91 | 73 |

*At a later stage in the development process the dissolution method proved to be misleading by giving too high values. The corrected values are listed in the last column.

The results are in accordance with the results obtained in example 2, thus showing that fast dissolution in an acidic solution is also achieved with co-milling of lornoxicam and sodium carbonate.

### Example 4

### The Effect of Co-Milling of Lornoxicam and Arginine on the Dissolution of Lornoxicam in an Acidic Solution

In the Present Example the Co-Milling is Performed by Means of a Ball Mill with Horizontally Moving Spheres.

Ingredients:

| | Ingredients | % (w/w) | |
|---|---|---|---|
| 1 | Lornoxicam | 2.0 | |
| 2 | Arginine | 18.7 | |
| 3 | Cellulose microcrystalline, | 20.8 | |
| | type 102 | | |
| 4 | Calcium monohydrogen phosphate, | 47.8 | |
| | dihydrate | | |
| | CaHPO₄, 2H₂O | | |
| 5 | Low substituted hydroxypropyl cellulose | 10.4 | |
| 6 | Calcium Stearate | 0.3 | |
| | Tablet weight (mg) | 313 | |
| | Particle size of the alkaline substance | 187 | μm |
| | D(v; 0.5) (μm) | | |

The amount of lornoxicam and arginine is in a molar relationship of 1:20.

Tablets were made as described in example 2 and dissolution testing was carried out according to example 1.

TABLE 4
Dissolution of the obtained tablets

| | % (w/w) dissolved |
|---|---|
| Type of alkaline substance | lornoxicam at 20 minutes |
| Arginine | 78.0 |

As can be seen from table 4 when comparing with example 9 (prior process without the wet-granulation step), a significant increase in the amount dissolved at 20 min. can be achieved when co-milling lornoxicam and arginine by use of a ball mill with horizontally moving spheres.

### Example 5

### The Effect of Mechano Fusion as the Co-Milling Method on the Dissolution of Lornoxicam in an Acidic Solution

Ingredients:

| | | Batch | Batch | Batch | Batch | Batch |
|---|---|---|---|---|---|---|
| | | 17030531 % | 17030532 % | 17030534 % | 17030537 % | 17030538 % |
| | Ingredients | (w/w) | (w/w) | (w/w) | (w/w) | (w/w) |
| 1 | Lornoxicam | 1.7 | 1.7 | 2.8 | 2.6 | 2.3 |
| 2 | Sodium carbonate, | | | 17.9 | | |
| | Na₂CO₃ | | | | | |
| 2 | Trisodium phosphate | 19.0 | 19.0 | | | |
| | Na₃PO₄ | | | | | |
| 2 | Arginine | | | | 18.1 | |
| 2 | Lysine | | | | | 18.4 |
| 3 | Cellulose microcrystalline, | 20.8 | 20.8 | 20.8 | 20.8 | 20.8 |
| | type 102 | | | | | |
| 4 | Calcium monohydrogen | 47.8 | 47.8 | 47.8 | 47.8 | 47.8 |
| | phosphate, dihydrate | | | | | |
| | CaHPO₄, 2H₂O | | | | | |
| 5 | Low substituted hydroxypropyl | 10.4 | 10.4 | 10.4 | 10.4 | 10.4 |
| | cellulose | | | | | |
| 6 | Calcium Stearate | 0.3 | 0.3 | 0.3 | 0.3 | 0.3 |
| | Tablet weight (mg) | 314 | 306 | 278 | 298 | 244 |
| | Mean particle size of the alkaline | 203 | 40 | 48 | 68 | 46 |
| | substance | | | | | |
| | D(v; 0.5) (μm) | | | | | |

The amount of lornoxicam and the alkaline substance is in a molar relationship of app. 1:20

Prior to the mechano fusion the alkaline substance (2) was milled by means of a Alpine®AS spiral jet mill from Hosokawa Alpine. The resulting mean particle size was determined and listed in the table above.

Co-milling of the alkaline substance (2) and lornoxicam (1) was performed with a AMS-LAB mechano fusion unit from Hosokawa Alpine. The parameters were as follows:
Time: 3-30 min
Rotor speed: 1300-1500
Temp: 20-45° C.

The rest of the ingredients listed (3) to (6) were admixed and tablets with a diameter of 10 mm were compressed. Dissolution testing according to example 1 was carried out.

TABLE 5
Dissolution results of resulting tablets

| | | % (w/w) dissolved | Mean |
|---|---|---|---|
| Type of alkaline | | lornoxicam at 20 | particle size |
| substance | Batch no. | minutes | D(v; 0.5) (μm) |
| Trisodium phosphate, | 17030531 | 57.0 | 203 |
| Na₃PO₄ | | | |
| Trisodium phosphate, | 17030532 | 67.2 | 40 |
| Na₃PO₄ | | | |
| Sodium carbonate, | 17030534 | 63.2 | |
| Na₂CO₃ | | | |
| Arginine | 17030537 | 62.3 | |
| Lysine | 17030538 | 80.1 | |

From table 5 is seen that co-milling by mechano fusion of lornoxicam and the alkaline substance lysine results in a significant increase in the amount dissolved at 20 minutes when comparing with example 9 (prior process without the wet-granulation step). When using arginine, trisodlum phosphate or sodium carbonate a positive impact on the dissolution can also be seen. Furthermore, the results illustrate that when using mechano fusion a small mean particle size of the alkaline substance has a positive impact on the dissolution. This is illustrated by the results achieved with co-milled mixtures containing trisodlum phosphate where the batch having the small mean particle size of the alkaline substance has a faster dissolution than the one having the relatively large mean particle size.

### Example 6

### The Effect of Using Roller Compaction as the Method of Co-Milling on the Dissolution of Lornoxicam in an Acidic Solution

Ingredients:

| | | Batch | Batch | Batch | Batch | Batch |
|---|---|---|---|---|---|---|
| | | 13050534 | 13050535 | 13050543 | 13060531 | 13060534 |
| | Ingredients | % (w/w) | % (w/w) | % (w/w) | % (w/w) | % (w/w) |
| 1 | Lornoxicam | 1.7 | 1.7 | 2.3 | 2.3 | 2.3 |
| 2 | Trisodium phosphate | 19.0 | 19.0 | | | |
| | Na₃PO₄ | | | | | |
| 2 | Lysine | | | 18.4 | 18.4 | 18.4 |
| 3 | Cellulose | 20.8 | 20.8 | 20.8 | 20.8 | 20.8 |
| | microcrystalline, | | | | | |
| | type 102 | | | | | |
| 4 | Calcium | 47.8 | 47.8 | 47.8 | 47.8 | 47.8 |
| | monohydrogen | | | | | |
| | phosphate, dihydrate | | | | | |
| | CaHPO₄, 2H₂O | | | | | |
| 5 | Low substituted | 10.4 | 10.4 | 10.4 | 10.4 | 10.4 |
| | hydroxypropyl | | | | | |
| | cellulose | | | | | |
| 6 | Calcium Stearate | 0.3 | 0.3 | 0.3 | 0.3 | 0.3 |
| | Tablet weight (mg) | 336 | 318 | 261 | 314 | 324 |
| | Mean particle size of | 24 μm | 203 μm | 158 μm | 26 μm | 158 μm |
| | the alkaline | | | | | |
| | substance | | | | | |
| | D(v; 0.5) (μm) | | | | | |

The amount of lornoxicam and the alkaline substance is in a molar relationship of approx. 1:20

Prior to roller compaction the alkaline substance (2) used in batches 13050534 and 13060531 was milled using a Fritz Pulverlsette (type 14.702). The resulting mean particle sizes are listed in the table above.

Roller compaction of the alkaline substance (2) and lornoxicam (1) was carried out using a Minipactor® from Gerteis Maschinen+Processengineering AG. The parameters were as follows:

Compaction force: 8-12 kN/cm

### Rpm: 2

Sieve size: 1.0-1.5 mm
Gab size 2.5 mm

The rest of the ingredients listed above (3) to (6) were admixed and tablets having a diameter of 10 mm were compressed. Dissolution testing according to example 1 was carried out.

TABLE 6
Dissolution of resulting tablets

| | | % (w/w) dissolved lornoxicam |
|---|---|---|
| Type of alkaline substance | Batch no. | at 20 minutes |
| Trisodium phosphate | 13050534 | 59.2 |
| Na₃PO₄ | | |
| Trisodium phosphate | 13050535 | 57.6 |
| Na₃PO₄ | | |
| Lysine | 13050543 | 89.5 |
| Lysine | 13060531 | 79.3 |
| Lysine | 13060534 | 82.3 |

From table 6 is seen that by performing co-milling by means of roller compaction the use of lysine as alkaline substance leads to a significant increase in amount dissolved at 20 min when comparing with the results of example 9 (prior process without the wet-granulation step). When using trisodlum phosphate as the alkaline substance a positive impact on the dissolution can also be seen. The difference in mean particle size of the alkaline substance has no major impact on dissolution when roller compaction is used as the method of co-milling.

### Example 7

### The Effect on the Dissolution of Lornoxicam in Tablet where the Alkaline Substance was Micronized Prior to Simple Admixing Followed by Compression into a Tablet

Ingredients

| | | Batch | Batch |
|---|---|---|---|
| | | 02060531 | 02060532 |
| | Ingredients | % (w/w) | % (w/w) |
| 1 | Lornoxicam | 2.3 | 1.2 |
| 2 | Lysine | 18.4 | 19.5 |
| 3 | Cellulose microcrystalline, | 20.8 | 20.8 |
| | type 102 | | |
| 4 | Calcium monohydrogen | 47.8 | 47.8 |
| | phosphate, dihydrate, | | |
| | CaHPO₄, 2H₂O | | |
| 5 | Low substituted hydroxypropyl | 10.4 | 10.4 |
| | cellulose | | |
| 6 | Calcium Stearate | 0.3 | 0.3 |
| | Tablet weight (mg) | 300 | 429 |
| | Mean particle size of the alkaline | 5.0 | 5.0 |
| | substance | | |
| | D(v; 0.5) (μm) | | |

The amount of lornoxicam and the alkaline substance is in a molar relationship of app. 1:20 for batch 02060531 and approx. 1:40 for batch 02060532.

The alkaline substance (2) was micronized by use of a Alpine® AS spiral jet mill from Hosokawa Alpine.

The alkaline substance (2) and the lornoxicam (1) were mixed by use of a blender (with propeller-like blades at the bottom).

Thereafter, the rest of the ingredients (3) to (6) were admixed by use of a tumble mixer and tablets having a diameter of 10 mm were compressed. Dissolution testing according to example 1 was carried out.

TABLE 7
Dissolution results of resulting tablets

| Type of | | % (w/w) dissolved |
|---|---|---|
| alkaline | | lornoxicam at |
| substance | Batch no. | 20 minutes |
| Lysine | 02060531 | 82.0 |
| Lysine | 02060532 | 82.9 |

From table 7 is seen that blending of micronized lysine and lornoxicam leads to a significant increase in the amount dissolved at 20 min when compared to example 9 (prior process without the wet-granulation step). An increase in the molar relationship of lysine, changing the ratio between lornoxicam and lysine from 1:20 to 1:40 does not have any major impact on the dissolution.

### Example 8

### The Effect on the Dissolution of Lornoxicam from Tablets where the Alkaline Substance was Subjected to Milling Prior to Compressing the Mixture of Ingredients into Tablets

### The Dissolution is Performed in an Acidic Solution.

Ingredients

| | | Milled mixture |
|---|---|---|
| | Ingredients | % (w/w) |
| 1 | Lornoxicam | 1.7 |
| 2 | Trisodium phosphate, dried* | 19.0 |
| | Na₃PO₄ | |
| 3 | Cellulose microcrystalline, type 102 | 20.8 |
| 3 | Cellulose microcrystalline, type 101 | |
| 4 | Calcium monohydrogen phosphate dihydrate | 47.8 |
| | CaHPO₄, 2H₂O | |
| 4 | Calcium monohydrogen phosphate anhydrous | |
| | CaHPO₄ | |
| 5 | Low substituted hydroxypropyl cellulose | 10.4 |
| 5 | Hydroxypropylcellulose | |
| 6 | Calcium stearate | 0.3 |
| | Tablet weight (mg) | 311 |
| | Mean particle size of alkaline substance | 79 |
| | D(v; 0.5) (μm) | |

*Na₃PO₄, 12H₂O were dried to a content of water less than approx. 2% (w/w). The amount of lornoxicam and the alkaline substance is in a molar relationship of approx. 1:20.

The dried trisodium phosphate (2) was milled using a Fritsch Pulverisette (type 06.002.00; a ball mill with horizontally moving spheres) and mixed with lornoxicam (1) by hand.

Thereafter, the rest of the excipients (3) to (6) were admixed in a tumble mixer. From the mixture of ingredients (1) to (6) were compressed tablets having a diameter of 10 mm.

Dissolution testing according to example 1 was carried out.

TABLE 8
Dissolution results of the obtained tablets

| | % (w/w) dissolved lornoxicam at 20 |
|---|---|
| Alkaline substance | minutes |
| Trisodium phosphate, | 53.1 |
| dried* | |
| Na₃PO₄ | |

*Na₃PO₄, 12H₂O were dried to a content of water less than app. 2% (w/w).

From table 8 is seen that prior milling of the alkaline material, trisodium phosphate, before compressing the mixture of ingredients into tablets, improves the amount dissolved at 20 minutes when compared to example 9.

### Example 9

### The Impact of Abandoning Wet-Granulation in the Manufacturing Process while Otherwise Performed According to EP 1109534

Ingredients

| | Ingredients | % (w/w) |
|---|---|---|
| 1 | Lornoxicam | 2.5 |
| 2 | Sodium bicarbonate | 12.5 |
| | NaHCO₃ | |
| 3 | Cellulose microcrystalline, | 30.0 |
| | type 101 | |
| 4 | Calcium monohydrogen phosphate, | 34.5 |
| | anhydrous | |
| | CaHPO₄ | |
| 5 | Low substituted hydroxylpropyl cellulose | 15.0 |
| 5 | Hydroxpropyl cellulose | 5.0 |
| 6 | Calcium Stearate | 0.5 |
| | Tablet weight (mg) | 320 |

The amount of lornoxicam and the alkaline substance is in a molar relationship of approx. 1:20.

The ingredients (2) to (5) were mixed using a Diosna high shear mixer. Thereafter, the lornoxicam (1) was admixed lege artis using a planetary mixer. Finally the calcium stearate (6) was admixed using a high shear mixer.

Tablets were compressed using a 10 mm round standard concave tablet design. Dissolution testing according to example 1 was carried out

TABLE 9
Dissolution results of the obtained tablets

| | % (w/w) dissolved lornoxicam at 20 |
|---|---|
| Alkaline substance | minutes |
| Sodium bicarbonate, | 31.3 |
| NaHCO₃ | |

From table 9 is seen that the dissolution result of the obtained tablets where the wet-granulation step is left out is below 50%. The obtained dissolution is clearly not satisfactory.

### Example 10

### The Impact of Co-Milling of Lornoxicam and the Alkaline Substance on the Chemical Stability of Lornoxicam

The Co-Milling is Performed with Ball Milling with Horizontally Moving Spheres.

To compare the chemical stability of compositions produced according to the invention with compositions produced by means of wet-granulation, the following batches were provided:

Tablets according to the invention, batch nos: 17110431 and 17110432

Tablets manufactured according to the invention comprising co-milling of the alkaline substance and lornoxicam were manufactured according to examples 2 and 3 with the below described variations:

Ingredients:

| | | Wet | Co- | |
|---|---|---|---|---|
| | | granulation | milling, | Co-milling |
| | | Batch | Batch | Batch |
| | | 10225671 | 17110431 | 17110432 |
| | Ingredients | % (w/w) | % (w/w) | % (w/w) |
| 1 | Lornoxicam | 2.5 | 2.4 | 1.7 |
| 2 | Sodium bicarbonate | 12.5 | | |
| | NaHCO₃ | | | |
| 2 | Sodium carbonate, dried* | | 15.6 | |
| | Na₂CO₃ | | | |
| 2 | Trisodium phosphate, | | | 19.0 |
| | dried** Na₃PO₄ | | | |
| 3 | Cellulose microcrystalline, | 30.0 | | |
| | type 101 | | | |
| 3 | Cellulose microcrystalline, | | 21.5 | 20.7 |
| | type 102 | | | |
| 4 | Calcium monohydrogen | 34.5 | | |
| | phosphate, anhydrous | | | |
| | CaHPO₄ | | | |
| 4 | Calcium monohydrogen | | 49.4 | 47.8 |
| | phosphate dihydrate | | | |
| | CaHPO₄, 2H₂O | | | |
| 5 | Low substituted | 15.0 | 10.7 | 10.4 |
| | hydroxypropyl cellulose | | | |
| 5 | Hydroxypropylcellulose | 5.0 | | |
| 6 | Calcium Stearate | 0.5 | 0.4 | 0.4 |
| | Tablet weight (mg) | 320 | 329 | 351 |

*Na₂CO₃, 10H₂O were dried to a content of water less than app. 2% (w/w).
**Na₃PO₄, 12H₂O were dried to a content of water less than app. 2% (w/w)

The amount of lornoxicam and the alkaline substance is in a molar relationship of approx. 1:20.

Co-milling of lornoxicam (1) together with the alkaline substance (2) was carried out for 10 min by use of a Fritsch Pulverisette (type 06.002.00; a ball mill with horizontally moving spheres). The rest of the ingredients listed above (3) to (6) were admixed and tablets having a diameter of 10 mm were compressed using a standard concave punch design. From the mixture of ingredients (1) to (6) were compressed tablets using a 10 mm round standard concave punch design.

Tablet produced with wet-granulation, batch no: 10225671

The tablets were manufactured according to EP 1109534: The tablets based on wet granulation were compressed using a 9.5 mm round standard concave punch design.

Furthermore, tablets based on wet granulation were coated as also described in EP 1109534.

### Stability Program

A stability program was performed, including the following batches:

Batch no: 10225671, Xefo Rapid, wet-granulation
Batch no: 17110431, co-milling
Batch no: 17110432, co-milling

The co-milled batches were compared to a batch, which was produced by means of wet-granulation. The degradation product of lornoxicam, HN-10004 was chosen as stability indicating parameter.

All three batches were packed and stored in alu-alu bags.

Condition of storage:

25° C./60% RH for six months
30° C./65% RH for six months
40° C./75% RH for six months

### CONCLUSION

At all testpoints the amount of HN-10004 in the batch nos. 17110431 and 17110432 (co-milled) was lower than in batch no. 10225671 (wet-granulated).

At the starting point of the test period, the water-content of the tablets were determined and found to be on the same level. At all the test points, the stability of the two co-milled batches were superior to a significant extent over the wet-granulated batch, despite the fact that the wet-granulated batch has a lower water-content.

From the results of the stability testing it is concluded that the use of co-milling leads to a composition having a significantly slower development in the formation of critical degradation products.

### Results

At the starting point the water-content of the tablets were determined by Loss on Drying (LOD) (30 min, 70° C.). The results are shown in table 10 below:

TABLE 10
Water content in the resulting tablets.

| | Water content (LOD) |
|---|---|
| Batch no | % (w/w) |
| 10225671 | 1.2 |
| 17110431 | 1.5 |
| 17110432 | 1.6 |

| | Batch no: | | |
|---|---|---|---|
| | 10225671 | Batch no: 17110431 | Batch no: 17110432 |
| | HN-10004 in | HN-10004 in | HN-10004 in |
| Storage time | % (w/w) of | % (w/w) of | % (w/w) of |
| (months) | lornoxicam | lornoxicam | lornoxicam |
| | 25° C./60% RH | | |
| 0 | 0.073 | 0.012 | 0.010 |
| 1.5 | 0.11 | 0.012 | 0.032 |
| 3 | 0.13 | 0.024 | 0.013 |
| 4.5 | 0.15 | 0.033 | 0.030 |
| 6 | 0.17 | 0.036 | 0.035 |
| | 30° C./65% RH | | |
| 0 | 0.073 | 0.012 | 0.010 |
| 1.5 | 0.047 | 0.015 | 0.014 |
| 3 | 0.17 | 0.037 | 0.024 |
| 4.5 | 0.18 | 0.047 | 0.044 |
| 6 | 0.22 | 0.051 | 0.053 |
| | 40° C./75% RH | | |
| 0 | 0.073 | 0.012 | 0.010 |
| 3 | 0.22 | 0.072 | 0.090 |
| 6 | 0.22 | 0.12 | 0.11 |

Method of analysis (HPLC):
Stationary phase: ODS, 5 μm, 100×2.1 mm.
Column temperature: 35° C.
Mobile phase: Solvent A: Dissolve 50.0 g ammonium acetate in 5000.0 mL Milli-Q-water, and add 8.0 mL tetrabutyl ammonium hydroxide (1.5M in water) and 22.0 mL methanol.
   Solvent B: Add 1.00 mL octylamine to 1000.00 mL acetonitrile.
   Gradient:

| Time | Flow | | |
|---|---|---|---|
| (min.) | (mL/min.) | Solvent A % | Solvent B % |
| 0 | 0.5 | 97 | 3 |
| 32 | 0.5 | 70 | 30 |
| 36 | 0.5 | 70 | 30 |
| 41 | 0.5 | 97 | 3 |
| 50 | 0.5 | 97 | 3 |

Autosampler temperature: 20° C.
Injection volume: 20 μL

### Detection: 280 nm

Runtime: 50 minutes

The relative retention time for HN-10004, calculated with respect to the principal peak, is approximately 0.77 and the relative response factor is 0.8.

### Example 11

### The Effect of Co-Milling on the Dissolution of Lornoxicam. In the Present Example the Co-Milling is Performed by Means of Ball Milling with Vertically Moving Spheres

Ingredients

| | | Batch | Batch | Batch | Batch |
|---|---|---|---|---|---|
| | | 07020533 | 07020534 | 19040531 | 03050532 |
| | Ingredients | % (w/w) | % (w/w) | % (w/w) | % (w/w) |
| 1 | Lornoxicam | 2.3 | 1.7 | 1.2 | 1.2 |
| 2 | Trisodium | | 19.0 | | |
| | phosphate, dried*) | | | | |
| | Na₃PO₄ | | | | |
| 2 | Lysine | 18.4 | | 19.5 | 19.5 |
| 3 | Cellulose | 20.8 | 20.8 | 20.8 | 20.8 |
| | microcrystalline, | | | | |
| | type 102 | | | | |
| 4 | Calcium | 47.8 | 47.8 | 47.8 | 47.8 |
| | monohydrogen | | | | |
| | phosphate, dihydrate | | | | |
| | CaHPO₄, 2H₂O | | | | |
| 5 | Low substituted | 10.4 | 10.4 | 10.4 | 10.4 |
| | hydroxypropyl | | | | |
| | cellulose | | | | |
| 6 | Calcium Stearate | 0.3 | 0.3 | 0.3 | 0.3 |
| | Tablet weight (mg) | 238 | 318 | 277 | 420 |
| | Mean particle size | 158 | — | 158 | 26 |
| | of alkaline | | | | |
| | substance | | | | |
| | D(v; 0.5) (μm) | | | | |

*)Na₃PO₄, 12H₂O were dried to a content of water less than app. 2% (w/w)

The amount of lornoxicam and the alkaline substance is in a molar relationship of approx. 1:20 for batch 07020533, 07020534 and approx. 1:40 for batch 19040531 and 03050532. Lornoxicam (1) and lysine (2) were co-milled using a Struers ball mill for 4-10 hours and 250-400 rpm followed by a sieving through a 700 μm mesh. Thereafter, the rest of the excipients (3) to (6) were admixed and tablets were compressed using a round 10 mm standard concave tablet design. Dissolution testing according to example 1 was carried out.

TABLE 11
Dissolution results of the obtained tablets.

| | | % (w/w) |
|---|---|---|
| Alkaline substance | Batch no. | dissolved lornoxicam at 20 min. |
| Lysine | 07020533 | 80.8 |
| Trisodium phosphate | 07020534 | 66.2 |
| Lysine | 19040531 | 85.6 |
| Lysine | 03050532 | 82.7 |

From table 11 can be seen that the use of a ball mill with vertically moving spheres results in a significantly improved amount of lornoxicam dissolved at 20 min when compared to example 9 (prior process without the wet-granulation step). An increase in the molar relationship between lornoxicam and lysine from 1:20 to 1:40 does not have any major Impact on the dissolution.

### Example 12

Examples of compositions to be manufactured by co-milling by means of roller compaction of lornoxicam, the alkaline substance and selected other excipients followed by admixture of the rest of the excipients and compressing into tablets.

Ingredients

| | | % | % | % | % | % | % |
|---|---|---|---|---|---|---|---|
| | Ingredients | (w/w) | (w/w) | (w/w) | (w/w) | (w/w) | (w/w) |
| 1 | Lornoxicam | 1-3 | 1-3 | 1-3 | 1-3 | 1-3 | 1-3 |
| 2 | Lysine | 9-20 | 9-20 | 9-20 | 9-20 | 9-20 | 9-20 |
| | Molar relationship, | 1:10-1:40 | 1:10-1:40 | 1:10-1:40 | 1:10-1:40 | 1:10-1:40 | 1:10-1:40 |
| | lornoxicam:lysine | | | | | | |
| 3 | Calcium monohydrogen | 10-50 | | | | | |
| | phosphate, anhydrous | | | | | | |
| | CaHPO₃ | | | | | | |
| 3 | Trisodium phosphate | | 10-50 | | | | |
| | Na₃PO₄ | | | | | | |
| 3 | Calcium Carbonate | | | 10-50 | | | |
| | CaCO₃ | | | | | | |
| 3 | Magnesium Aluminium | | | | 10-50 | | |
| | Silicate | | | | | | |
| 3 | Magnesium oxide | | | | | 10-50 | |
| 3 | Calcium sulphate dihydrate | | | | | | 10-50 |
| | CaSO₄, 2H₂O | | | | | | |
| 4 | Sorbitol | | | 5-15 | | | |
| 5 | Talc | 0-10 | 0-10 | 0-10 | 0-10 | 0-10 | 0-10 |
| 6 | Cellulose microcrystalline, | 15-25 | 15-25 | 15-25 | 15-25 | 15-25 | 15-25 |
| | type 102 | | | | | | |
| 7 | Low substituted | 5-15 | 5-15 | 5-15 | 5-15 | 5-15 | 5-15 |
| | hydroxypropyl cellulose | | | | | | |
| 8 | Calcium Stearate | 0.2-1.0 | 0.2-1.0 | 0.2-1.0 | 0.2-1.0 | 0.2-1.0 | 0.2-1.0 |

The ingredients (1) to (2) are premixed, sieved and mixed. The mean particle size of (2) is ranging between a D(v;0.5) of 5 μm to 160 μm. Thereafter, ingredient (5) is admixed following by admixture of (3) and (4). This mixture is co-milled by use of methods like roller compaction, ball milling (both horizontally and vertically moving spheres) or mixing in a blender. Thereafter the rest of the ingredients (6) to (8) are combined with the mixture of ingredients (1) to (5) by admixing. The combined mixture of the ingredients (1) to (8) is compressed into tablets.

The Individual amounts are adjusted so that each composition contains 4-12 mg lornoxicam, the molar ratio of lornoxicam to alkaline substance is in the range of 1:10 to 1:40 and so that the total amount of ingredients does not exceed 100%.

Tablets having a diameter of 10 mm are compressed by use of punch design of a round standard concave. The tablets can be coated afterwards in order to obtain coloured or white or moisture protected tablets as described in EP 1109534.

What is claimed is:

1. A process for manufacturing a pharmaceutical composition comprising the steps of:
a) providing an active drug substance, which has a solubility at room temperature of less than 0.1% w/v in 0.1 N hydrochloric acid or has a pKa value of less than 5.5;
b) providing one or more alkaline substance(s); and
c) mixing said active drug substance and said alkaline substance(s) by co-milling without adding a liquid; and optionally
d) admixing one or more pharmaceutically acceptable excipients; and optionally
e) compressing said mixture c) or d) into a tablet.

2. The process according to claim 1, wherein the co-milling step (c) is applied only to the active drug substance and the alkaline substance(s).

3. The process according to claim 1 wherein said process is undertaken under dry conditions excluding the use of liquid.

4. The process according to claim 1, wherein the composition comprises 4-12 mg lornoxicam.

5. The process according to claim 1, wherein said composition is in the form of a compressed tablet.

6. The process according to claim 1, wherein said co-milling is provided by a roller compactor.

7. The process according to claim 1, wherein the molar ratio of the active drug substance and the alkaline substance is between 1:100 and 1:1.

8. The process according to claim 1, wherein the molar ratio of the active drug substance and the alkaline substance(s) is between 1:100 and 1:10.

9. The process according to claim 1, wherein the molar ratio of the active drug substance and the alkaline substance(s) is/are in the range of 1:10 to 1:40, and wherein said active drug substance is lornoxicam.

10. The process according to claim 1, wherein the alkaline substance is water soluble as characterised by that 1 part of the alkaline substance is soluble in a maximum of 100 parts of water.

11. The process according to claim 1, wherein the alkaline substance is a salt of an organic acid, a salt of an inorganic acid, an organic amine or an amino acid or a derivative thereof.

12. The process according to claim 11, wherein the alkaline substance is an amino acid or a derivative thereof.

13. The process according to claim 11, wherein the amino acid or a derivative thereof is lysine, arginine or histidine.

14. The process according to claim 11, wherein the organic acid and the inorganic acid has a pKa in the range of 4-14.

15. The process according to claim 11, wherein the alkaline substance is a salt of an inorganic acid selected from carbonic acid or phosphoric acid.

16. The process according to claim 11, wherein the active drug substance is an NSAID or a pharmaceutically acceptable salt or a prodrug thereof.

17. The process according to claim 16, wherein the NSAID is ampiroxicam, droxicam, lornoxicam, meloxicam, piroxicam, tolfenamic acid or tenoxicam or a pharmaceutically acceptable salt or prodrug thereof.

18. The process according to claim 16, wherein the NSAID is lornoxicam or a pharmaceutically acceptable salt or prodrug thereof.

19. The process according to claim 16, wherein the molar ratio of lornoxicam and the alkaline substance(s) is/are between 1:100 and 1:10.

20. The process according to claim 16, wherein the NSAID is lornoxicam and wherein the amino acid or a derivative thereof is lysine.
